(19) United States
(12) Patent Application Publication
(10) Pub. No.: US 2003/0207923 A1
(43) Pub. Date: Nov. 6, 2003

(54) 2,'3-Amino-4-(n-hydroxyamino)-succinylamino-acetamides for use as cd23 formation inhibitors

(76) Inventors: Andrew Faller, Epping Essex (GB); Anne-Geraldine Rousseau, King of Prussia, PA (US); David Timothy Macpherson, Dunmow (GB); Peter Henry Milner, Dunmow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/169,119

(22) PCT Filed: Dec. 22, 2000
(86) PCT No.: PCT/GB00/05012

(30) Foreign Application Priority Data
Dec. 29, 1999 (GB) 9930754.8

(51) Int. Cl. A61K 31/44; C07C 239/18; A61K 31/19; C07C 259/04

(52) U.S. Cl. 514/357; 514/575; 562/623; 546/336

(57) ABSTRACT

Compounds of formula (I): wherein X¹ is alkyl, sulphonyl or carboxy: X² is hydrogen or alkyl; R¹ is arylmethyl or heterocyclylmethyl; R² is alkyl, alkenyl, aryl, cycloalkyl or cycloalkenyl; and R³ is hydrogen, alkyl, alkenyl, alkynyl or aryl; are useful in the treatment of disorders mediated by s-CD23.

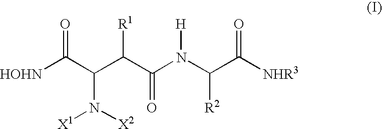

This invention relates to novel inhibitors of the formation of soluble human CD23 and their use in the treatment of conditions associated with excess production of soluble CD23 (s-CD23) such as autoimmune disease, inflammation and allergy. The compounds of the invention are also inhibitors of the release of tumour necrosis factor (TNF).

CD23 (the low affinity IgE receptor FceRII, Blast 2), is a 45 kDa type II integral protein expressed on the surface of a variety of mature cells, including B and T lymphocytes, macrophages, natural killer cells, Langerhans cells, monocytes and platelets (Delespesse et al, Adv. Immunol, 49 [1991] 149-191). There is also a CD23-like molecule on eosinophils (Grangette et al, J Immunol, 143 [1989] 3580-3588). CD23 has been implicated in the regulation of the immune response (Delespesse et al, Immunol Rev, 125 [1992] 77-97). Human CD23 exists as two differentially regulated isoforms, a and b, which differ only in the amino acids at the intracellular N-terminus (Yokota et al, Cell, 55 [1988] 611-618). In man the constitutive a isoform is found only on B-lymphocytes, whereas type b, inducible by IL-4, is found on all cells capable of expressing CD23.

Intact, cell bound CD23 (i-CD23) is known to undergo cleavage from the cell surface leading to the formation of a number of well-defined soluble fragments (s-CD23), which are produced as a result of a complex sequence of proteolytic events, the mechanism of which is still poorly understood (Bourget et al J Biol Chem, 269 [1994] 6927-6930). Although not yet proven, it is postulated that the major soluble fragments (Mr 37, 33, 29 and 25 kDa) of these proteolytic events, all of which retain the C-terminal lectin domain common to i-CD23, occur sequentially via initial formation of the 37 kDa fragment (Letellier et al, J Exp. Med., 172 [1990] 693-700). An alternative intracellular cleavage pathway leads to a stable 16 kDa fragment differing in the C-terminal domain from i-CD23 (Grenier-Brosette et al, Eur J Immunol, 22 [1992] 1573-1577).

Several activities have been ascribed to membrane bound i-CD23 in humans, all of which have been shown to play a role in IgE regulation. Particular activities include: a) antigen presentation, b) IgE mediated eosinophil cytotoxicity, c) B cell homing to germinal centres of lymph nodes and spleen, and d) down-regulation of IgE synthesis (Delespesse et al, Adv Immunol, 49, [1991] 149-191). The three higher molecular weight soluble CD23 fragments (Mr 37, 33 and 29 kDa) have multifunctional cytokine properties which appear to play a major role in IgE production. Thus, the excessive formation of s-CD23 has been implicated in the overproduction of IgE, the hallmark of allergic diseases such as extrinsic asthma, rhinitis, allergic conjunctivitis, eczema, atopic dermatitis and anaphylaxis (Sutton and Gould, Nature, 366, [1993] 421-428). Other biological activities attributed to s-CD23 include the stimulation of B cell growth and the induction of the release of mediators from monocytes. Thus, elevated levels of s-CD23 have been observed in the serum of patients having B-chronic lymphocytic leukaemia (Sarfati et al, Blood, 71 [1988] 94-98) and in the synovial fluids of patients with rheumatoid arthritis (Chomarat et al, Arthritis and Rheumatism, 36 [1993] 234-242). That there is a role for CD23 in inflammation is suggested by a number of sources. First, sCD23 has been reported to bind to extracellular receptors which when activated are involved in cell-mediated events of inflammation. Thus, sCD23 is reported to directly activate monocyte TNF, IL-1, and IL-6 release (Armant et al, vol 180, J. Exp. Med., 1005-1011 (1994)). CD23 has been reported to interact with the B2-integrin adhesion molecules, CD11b and CD11c on monocyte/macrophage (S. Lecoanet-Henchoz et al, Immunity, vol 3; 119-125 (1995)) which trigger NO⁻, hydrogen peroxide and cytokine (IL-1, IL-6, and TNF) release. Finally, IL-4 or IFN induce the expression of CD23 and its release as sCD23 by human monocytes. Ligation of the membrane bound CD23 receptor with IgE/anti-IgE immune complexes or anti CD23 mAb activates cAMP and IL6 production and thromboxane B2 formation, demonstrating a receptor-mediated role of CD23 in inflammation.

Because of these various properties of CD23, compounds which inhibit the formation of s-CD23 should have twofold actions of a) enhancing negative feedback inhibition of IgE synthesis by maintaining levels of i-CD23 on the surface of B cells, and b) inhibiting the immunostimulatory cytokine activities of higher molecular weight soluble fragments (Mr 37, 33 and 29 kDa) of s-CD23. In addition, inhibition of CD23 cleavage should mitigate sCD23-induced monocyte activation and mediator formation, thereby reducing the inflammatory response.

TNFα is a pro-inflammatory cytokine which is released from stimulated cells by specific cleavage of a 76-amino acid signal sequence in the inactive precursor to generate the mature form. The cleavage of TNFα has been reported to be carried out by a metalloprotease (Gearing, A. J. H. et al, (1994) Nature 370, 555-557; McGeehan, G. M. et al, (1994) Nature 370, 558-561; Mohler, K. M. et al, (1994) Nature 370, 218-220). Compounds reported to inhibit the cleavage of TNFα by the TNF processing enzyme can be broadly described as matrix metalloprotease inhibitors, particularly of the hydroxamic acid class.

TNFα is induced in a variety of cell types in response to bacteria, endotoxin, various viruses and parasites, so that one physiological function ascribed to TNFα is a contribution to the inflammatory response to acute infection by bacteria, parasites, etc (Dinarello, C. A. (1992) Immunol. 4, 133-145). Overproduction of TNFα has been implicated in disease states such as rheumatoid arthritis, septic shock, Crohn's disease and cachexia (Dinarello, 1992). inhibition of processing of TNFα to the mature, active form would therefore be beneficial in the treatment of these inflammatory disorders. TNFα may also contribute to the destruction of tissue in autoimmune disease although it is not a initiating factor in these diseases. Confirming the importance of TNFα in rheumatoid arthritis, TNFα antibodies have been shown to reduce the severity of disease in short term studies in rheumatoid arthritis models (Elliott, M. J., et al (1993) Arthrit. Rheum. 12, 1681-1690; Elliott et al (1994) Lancet 344, 1125-1127).

International Patent Application No. WO 96/02240 (SmithKline Beecham plc) discloses that compounds which inhibit the action of matrix metalloproteases (e.g. collagenase, stromelysin and gelatinase) are effective inhibitors of the release of human soluble CD23 transfected into mammalian cell culture systems.

International Patent Application No. WO 97/43249 (SmithKline Beecham plc) discloses that certain compounds of formula (A):

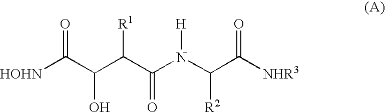

are effective inhibitors of CD23 processing and TNF release, whilst exhibiting reduced collagenase inhibitory activity.

According to the present invention there is provided a compound of formula (I):

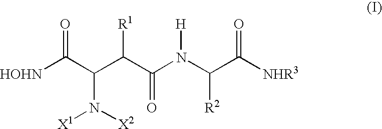

wherein:

X¹ is alkyl, sulphonyl, or carboxy;

X² is hydrogen or alkyl;

R¹ is arylmethyl or heterocyclylmethyl;

R² is alkyl, alkenyl, aryl, cycloalkyl or cycloalkenyl; and

R³ is hydrogen, alkyl, alkenyl, alkynyl or aryl.

Alkyl, sulphonyl, carboxy, alkenyl and alkynyl groups referred to herein include straight and branched groups containing up to six carbon atoms and are optionally substituted by one or more groups selected from the group consisting of aryl, heterocyclyl, (C₁₋₆)alkylthio, (C₁₋₆)alkoxy, aryl(C₁₋₆)alkenyl, aryl(C₁₋₆)alkoxy, aryl(C₁₋₆)alkylthio, amino, mono- or di-(C₁₋₆)alkylamino, cycloalkyl, cycloalkenyl, carboxy and esters thereof, hydroxy, and halogen.

Cycloalkyl and cycloalkenyl groups referred to herein include groups having between three and eight ring carbon atoms and are optionally substituted as described hereinabove for alkyl, alkenyl and alkynyl groups.

When used herein, the term “aryl” means single and fused rings suitably containing from 4 to 7, preferably 5 or 6, ring atoms in each ring, which rings, may each be unsubstituted or substituted by, for example, up to three substituents. A fused ring system may include aliphatic rings and need include only one aromatic ring.

Suitable aryl groups include phenyl and naphthyl such as 1-naphthyl or 2-naphthyl.

Suitably any aryl group, including phenyl and naphthyl, may be optionally substituted by up to five, preferably up to three substituents. Suitable substituents include halogen, (C₁₋₆)alkyl, aryl, aryl(C₁₋₆)alkyl, (C₁₋₆)alkoxy, (C₁₋₆)alkoxy(C₁₋₆)alkyl, halo(C₁₋₆)alkyl, aryl(C₁₋₆)alkoxy, hydroxy, nitro, cyano, azido, amino, mono- and di-N-(C₁₋₆)alkylamino, acylamino, arylcarbonylamino, acyloxy, carboxy, carboxy salts, carboxy esters, carbamoyl, mono- and di-N-(C₁₋₆)alkylcarbamoyl, (C₁₋₆)alkoxycarbonyl, aryloxycarbonyl, ureido, guanidino, sulphonylamino, aminosulphonyl, (C₁₋₆)alkylthio, (C₁₋₆)alkyl sulphinyl, (C₁₋₆)alkylsulphonyl, heterocyclyl and heterocyclyl (C₁₋₆)alkyl. In addition, two adjacent ring carbon atoms may be linked by a (C₃₋₅)alkylene chain, to form a carbocyclic ring.

When used herein the terms “heterocyclyl” and “heterocyclic” suitably include, unless otherwise defined, aromatic and non-aromatic, single and fused, rings suitably containing up to four heteroatoms in each ring, each of which is selected from oxygen, nitrogen and sulphur, which rings, may be unsubstituted or substituted by, for example, up to three substituents. Each heterocyclic ring suitably has from 4 to 7, preferably 5 or 6, ring atoms. A fused heterocyclic ring system may include carbocyclic rings and need include only one heterocyclic ring.

Preferably a substituent for a heterocyclyl group is selected from halogen, (C₁₋₆)alkyl, aryl(C₁₋₆)alkyl, (C₁₋₆)alkoxy, (C₁₋₆)alkoxy(C₁₋₆)alkyl, halo(C₁₋₆)alkyl, hydroxy, amino, mono- and di-N-(C₁₋₆)alkyl-amino, acylamino, carboxy salts, carboxy esters, carbamoyl, mono- and di-N-(C₁₋₆)alkylcarbonyl, aryloxycarbonyl, (C₁₋₆)alkoxycarbonyl(C₁₋₆)alkyl, aryl, oxy groups, ureido, guanidino, sulphonylamino, aminosulphonyl, (C₁₋₆)alkylthio, (C₁₋₆)alkylsulphinyl, (C₁₋₆)alkylsulphonyl, heterocyclyl and heterocyclyl(C₁₋₆)alkyl.

In a particular aspect of the invention, X¹ is sulphonyl and X² is hydrogen. In a further aspect of the invention, each of X¹ and X², and R¹ to R³ is selected from the group consisting of the values ascribed to it in the Examples hereinbelow. Preferably, the compound of formula (I) of the invention is selected from the group consisting of the compounds described in the Examples hereinbelow.

According to a further aspect, the present invention provides the use of a compound of formula (I) for the production of a medicament for the treatment or prophylaxis of disorders such as allergy, inflammatory disorders and autoimmune disease in which the overproduction of s-CD23 is implicated.

In a further aspect the invention provides a method for the treatment or prophylaxis of disorders such as allergy, inflammatory disorders and autoimmune disease in which the overproduction of s-CD23 is implicated, which method comprises the administration of a compound of formula (I), to a human or non-human mammal in need thereof.

The invention also provides a pharmaceutical composition for the treatment or prophylaxis of disorders such as allergy, inflammatory disorders and autoimmune disease in which the overproduction of s-CD23 is implicated which comprises a compound of formula (I) and optionally a pharmaceutically acceptable carrier therefor.

According to a further aspect, the present invention provides the use of a compound of formula (I) for the production of a medicament for the treatment or prophylaxis of conditions mediated by TNF, including, but not limited to, inflammation, fever, cardiovascular effects, haemorrhage, coagulation and acute phase response, cachexia and anorexia, acute infections, shock states, graft versus host reactions and autoimmune disease.

In a further aspect the invention provides a method for the treatment or prophylaxis of conditions mediated by TNF, which method comprises the administration of a compound of formula (I), to a human or non-human mammal in need thereof.

The invention also provides a pharmaceutical composition for the treatment or prophylaxis of conditions mediated by TNF, which corn comprises a compound of formula (I) and optionally a pharmaceutically acceptable carrier therefor.

Particular inflammatory disorders include CNS disorders such as Alzheimer's disease, multiple sclerosis, and multi-infarct dementia, as well as the inflammation mediated sequelae of stroke and head trauma.

It is to be understood that the pharmaceutically acceptable salts, solvates and other pharmaceutically acceptable derivatives of the compound of formula (I) are also included in the present invention.

Salts of compounds of formula (I) include for example acid addition salts derived from inorganic or organic acids, such as hydrochlorides, hydrobromides, hydroiodides, p-toluenesulphonates, phosphates, sulphates, acetates, trifluoroacetates, propionates, citrates, maleates, fumarates, malonates, succinates, lactates, oxalates, tartarates and benzoates.

Salts may also be formed with bases. Such salts include salts derived from inorganic or organic bases, for example alkali metal salts such as sodium or potassium salts, and organic amine salts such as morpholine, piperidine, dimethylamine or diethylamine salts.

It has surprisingly been found that the compounds of the present invention are potent and selective inhibitors of CD23 processing and TNF release, whilst exhibiting reduced collagenase inhibitory activity.

The compounds of the invention may be prepared by use of any appropriate conventional method, for example by analogy with the methods disclosed in patent publication WO 97/02239 (BBL), or by synthesis on a solid-phase support such as Wang hydroxylamine resin (Tetrahedron Lett. 37(44) [1996] 8045).

Accordingly, a further aspect of the invention provides a process for preparing a compound of formula (I) as defined hereinabove, which process comprises:

(a) deprotecting a compound of formula (II):

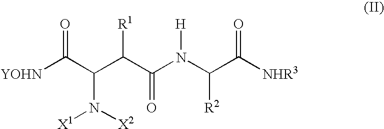

wherein X¹, X² and R¹ to R³ are as defined hereinabove, and Y is a protecting group such as benzyl or trimethylsilyl, or cleaving the compound of formula (II) from the Wang resin, where Y is the Wang resin residue, or

(b) reacting a compound of formula (III):

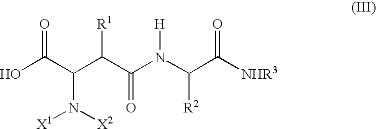

wherein X¹, X² and R¹ to R³ are as defined hereinabove, and any hydroxy group is optionally protected, with hydroxylamine or a salt thereof, or with Wang hydroxylamine resin followed by cleavage from the resin, or

(c) converting a compound of formula (I) to a different compound of formula (I) as defined hereinabove.

Compounds of formulae (II) and (III) are novel and form a further aspect of the invention.

Compounds of formula (II) where Y is a protecting group can be prepared from compounds of formula (III) by reaction with a protected hydroxylamine. Suitable protecting groups for a hydroxamic acid are well known in the art and include benzyl, trimethylsilyl, t-butyl and t-butyldimethylsilyl.

Compounds of formula (III) can be prepared by deprotecting a compound of formula (IV):

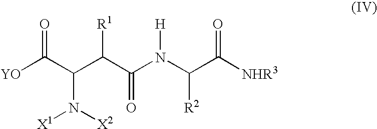

wherein X¹, X² and R¹ to R³ are as defined hereinabove and Y is a protecting group such as t-butyl, or cleaving the compound of formula (IV) from the Wang resin, where Y is the Wang resin residue.

Compounds of formula (II) where Y is a Wang residue can be prepared from compounds of formula (V):

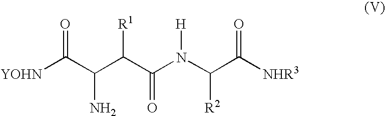

wherein Y and R¹ to R³ are as defined hereinabove, by reaction with a compound of formula X¹.Q, wherein X¹ is as defined hereinabove and Q is a leaving group, and thereafter optionally reacting with a compound of formula X².Q wherein X² and Q are as defined hereinabove.

Compounds of formula (V) can be prepared by cleavage of a compound of formula (VI):

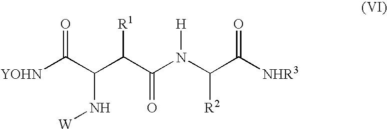

wherein Y and R¹ to R³ are as defined hereinabove and W is a protecting group such as an alloc group.

Compounds of formula (VI) can be prepared by attachment of a compound of formula (VII) to a Wang resin:

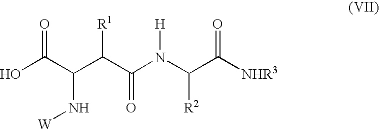

wherein R¹ to R² are as defined herein above.

Compounds of formula (VI) can be prepared by hydrolysis of a compound of formula (VIII):

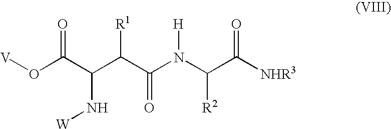

wherein R¹ to R³ and W are as defined hereinabove, and V is a protecting group such as t-butyl.

Suitable protecting groups for a hydroxamic acid are well known in the art and include benzyl, trimethylsilyl, t-butyl and t-butyldimethylsilyl.

Suitable protecting groups for a carboxylic acid are well known in the art and include benzyl, t-butyl and methyl.

Compounds of formula (VIII) can be prepared by reacting a compound of formula (IX):

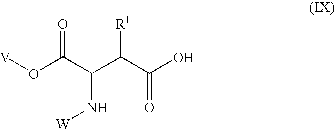

wherein V, W and R¹ are as defined hereinabove, with a compound of formula (IXa):

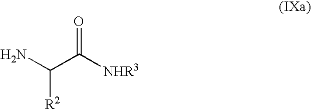

wherein R² and R³ are as defined hereinabove.

Compounds of formula (IX) can be prepared by hydrolysis of a compound of formula (X):

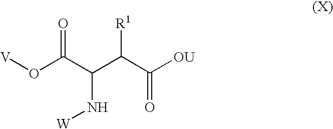

wherein V, W and R¹ are as defined hereinabove and U is a carboxylic acid protecting group such as methyl.

Compounds of formula (X) can be prepared from compounds of formula (XI):

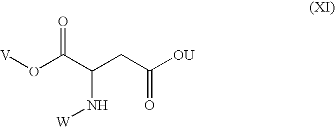

wherein V, and W are as defined hereinabove by reaction with a compound of formula R¹.Q wherein R¹ is as defined hereinabove and Q is a leaving group such as bromine.

Compounds of formula (XI) can be prepared by reaction of a compound of formula (XII):

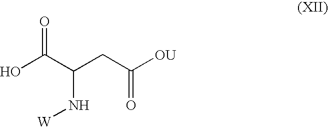

wherein W and U are as defined hereinabove with a compound of formula V.Q wherein Q is a leaving group.

Compounds of formula (XII) can be prepared from compounds of formula (XIII):

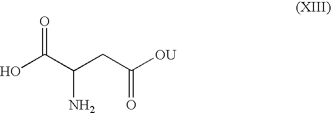

wherein U is as defined hereinabove by reaction with a compound of formula W.Q wherein Q is a leaving group.

The starting materials and other reagents are available commercially or can be synthesised by well-known and conventional methods.

The isomers, including stereoisomers, of the compounds of the present invention may be prepared as mixtures of such isomers or as individual isomers. The individual isomers may be prepared by any appropriate method, for example individual stereoisomers may be prepared by stereospecific chemical synthesis starting from chiral substrates or by separating mixtures of diastereoisomers using known methods. In a preferred aspect, the invention provides compounds of formula (IA):

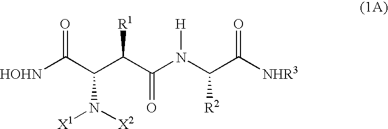

It is preferred that the compounds are isolated in substantially pure form.

As stated herein an inhibitor of the formation of soluble human CD23 has useful medical properties. Preferably the active compounds are administered as pharmaceutically acceptable compositions.

The compositions are preferably adapted for oral administration. However, they may be adapted for other modes of administration, for example in the form of a spray, aerosol or other conventional method for inhalation, for treating respiratory tract disorders; or parenteral administration for patients suffering from heart failure. Other alternative modes of administration include sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilising the compound and a sterile vehicle, and, depending on the concentration used, can be either suspended or dissolved in the vehicle. In preparing solutions the compound can be dissolved in water for injection and filter sterilised before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

Compositions of this invention may also suitably be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns for example diameters in the range of 1-50 microns, 1-10 microns or 1-5 microns. Where appropriate, small amounts of other anti-asthmatics and bronchodilators, for example sympathomimetic amines such as isoprenaline, isoetharine, salbutamol, phenylephrine and ephedrine; xanthine derivatives such as theophylline and arninophylline and corticosteroids such as prednisolone and adrenal stimulants such as ACTH may be included.

The compositions may contain from 0.1% to 99% by weight, preferably from 10-60% by weight, of the active material, depending upon the method of administration. A preferred range for inhaled administration is 10-99%, especially 60-99%, for example 90, 95 or 99%.

Micro fine powder formulations may suitably be administered in an aerosol as a metered dose or by means of a suitable breath-activated device.

Suitable metered dose aerosol formulations comprise conventional propellants, cosolvents, such as ethanol, surfactants such as oleyl alcohol, lubricants such as oleyl alcohol, desiccants such as calcium sulphate and density modifiers such as sodium chloride.

Suitable solutions for a nebulizer are isotonic sterilised solutions, optionally buffered, at for example between pH 4-7, containing up to 20 mg/ml of compound but more generally 0.1 to 10 mg/ml, for use with standard nebulisation equipment.

An effective amount will depend on the relative efficacy of the compounds of the present invention, the severity of the disorder being treated and the weight of the sufferer. Suitably, a unit dose form of a composition of the invention may contain from 0.1 to 1000 mg of a compound of the invention (0.001 to 10 mg via inhalation) and more usually from 1 to 500 mg, for example 1 to 25 or 5 to 500 mg. Such compositions may be administered from 1 to 6 times a day, more usually from 2 to 4 times a day, in a manner such that the daily dose is from 1 mg to 1 g for a 70 kg human adult and more particularly from 5 to 500 mg. That is in the range of about 1.4×10⁻² mg/kg/day to 14 mg/kg/day and more particularly in the range of about 7×10⁻² mg/kg/day to 7 mg/kg/day.

The following examples illustrate the invention but do not limit it in any way.

Biological Test Methods

Procedure 1: The ability of test compounds to inhibit the release of soluble CD23 was investigated by use of the following procedure.

RPMI 8866 Cell Membrane CD23 Cleavage Activity Assay:

Plasma membranes from RPMI 8866 cells, a human Epstein-Barr virus transformed B-cell line (Sarfati et al., Immunology 60 [1987] 539-547) expressing high levels of CD23 are purified using an aqueous extraction method. Cells resuspended in homogenisation buffer (20 mM HEPES pH 7.4, 150 mM NaCl, 1.5 MM MgCl2, 1 mM DTT) are broken by N2 cavitation in a Parr bomb and the plasma membrane fraction mixed with other membranes is recovered by centrifugation at 10,000×g. The light pellet is resuspended in 0.2 M potassium phosphate, pH 7.2 using 2 ml per 1-3 g wet cells and the nuclear pellet is discarded. The membranes are further fractionated by partitioning between Dextran 500 (6.4% w/w) and polyethylene glycol (PEG) 5000 (6.4% w/w) (ref), at 0.25 M sucrose in a total of 16 g per 10-15 mg membrane proteins [Morre and Morre, BioTechniques 7, 946-957 (1989)]. The phases are separated by brief centrifugation at 1000×g and the PEG (upper) phase is collected, diluted 3-5 fold with 20 mM potassium phosphate buffer pH 7.4, and centrifuged at 100,000×g to recover membranes in that phase. The pellet is resuspended in phosphate-buffered saline and consists of 34 fold enriched plasma membranes as well as some other cell membranes (e.g. lysosomes, Golgi). The membranes are aliquoted and stored at −80° C. Fractionation at 6.6% Dextran/PEG yields plasma membranes enriched 10-fold.

The fractionated membranes are incubated at 37° C. for times up to 4 hrs to produce fragments of CD23 which are separated from the membrane by filtration in 0.2 micron Durapore filter plates (Millipore) after quenching the assay with 5 uM Preparation 1 from P 30994. sCD23 released from the membrane is determined using the EIA kit from The Binding Site (Birmingham, UK) or a similar one utilising MHM6 anti-CD23 mAb [Rowe et al., Int. J. Cancer, 29, 373-382 (1982)] or another anti-CD23 mAb as the capture antibody in a sandwich EIA. The amount of soluble CD23 made by 0.5 ug membrane protein in a total volume of 50 ul phosphate-buffered saline is measured by EIA and compared to the amount made in the presence of various concentrations of inhibitors. Inhibitors are prepared in solutions of water or dimethylsulfoxide (DMSO) and the final DMSO concentration is not more than 2%. IC50's are determined by curve fitting as the concentration where 50% inhibition of production of sCD23 is observed relative to the difference in sCD23 between controls incubated without inhibitor.

Procedure 2: The ability of test compounds to inhibit collagenase was investigated using the following procedure.

Collagenase Inhibition Assay:

The potency of compounds to act as inhibitors of collagenase was determined by the method of Cawston and Barrett (Anal. Biochem. 99, 340-345, 1979), hereby incorporated by reference, whereby a 1 mM solution of the inhibitor being tested or dilutions thereof, was incubated at 37° C. for 18 h with collagen and human recombinant collagenase, from synovial fibroblasts cloned, expressed and purified from E. Coli, (buffered with 150 mM Tris, pH 7.6, containing 15 mM calcium chloride, 0.05% Brij 35, 200 mM sodium chloride and 0.02% sodium azide). The collagen was acetylated ³H type 1 bovine collagen prepared by the method of Cawston and Murphy (methods in Enzymology 80, 711,1981) The samples were centrifuged to sediment undigested collagen and an aliquot of the radioactive supernatant removed for assay on a scintillation counter as a measure of hydrolysis. The collagenase activity in the presence of 1 mM inhibitor, or dilution thereof, was compared to activity in a control devoid of inhibitor and the results reported as that concentration effecting 50% of the collagenase (IC₅₀).

Procedure 3: The ability of test compounds to inhibit TNF release was investigated using the following procedure.

Assay for Inhibition of Release of TNFα from Human Monocytes Stimulated by Lipopolysaccharide (LPS) Endotoxin.

Human monocytes, cultured in RPMI 1640 medium supplemented with 10% fetal calf serum, are centrifuged at 1000×g for 5 min and then resuspended in medium at 2×10⁶ cells/ml. The cell suspension is aliquoted in 24 well plates, 1 ml per well. Compounds to be tested are dissolved in neat dimethyl sulfoxide (DMSO) and added to culture with the final DMSO concentration at 0.1%. Compounds are added to cells in triplicate wells. TNF a release is stimulated by addition of LPS to the cells at a final concentration of 200 ng/ml. Appropriate control cultures are set up in triplicates also. The plates are incubated for 18-20 hrs at 37° C., 5% CO₂, then centrifuged at 1000×g for 5 min. A specific ELISA for human TNFα (SmithKline Beecham) is used to measure TNF levels in the cell-free culture supernatants.

Preparation of Resin Bound N′-[3S-amino-4-(N-hydroxyamino)-2R-(2-naphthylmethyl)succinyl]-S-tert-leucine-N-methylamide

Step 1

(S)-N-Allyloxycarbonylaspartic Acid β-Methyl Ester

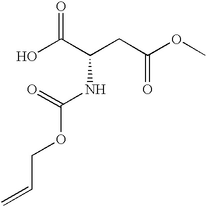

A mixture of aspartic acid β-methyl ester hydrochloride (36.8 g, 0.2 mol) and potassium carbonate (82.9 g, 0.6 mol) in a two phase mixture of diethyl ether (200 ml) and water (400 ml) was stirred vigorously at 0° C. and allyl chloroformate (28.9 g, 25.5 ml, 0.24 mol) was added dropwise over 20 minutes. The mixture was allowed to reach room temperature and stirred for 6 hrs. The layers were separated and the aqueous layer was washed with ether (3×) and then acidified to pH 2 with 2M HCl. The product was extracted into ethyl acetate and the extracts were washed with water and then dried (MgSO₄) and evaporated to give the title compound as a colourless gum. (38.4 g, 83%)

MS (APCI−ve) M−H=230

¹H NMR (CDCl₃) 2.88 (1H, dd, J=4.7, 17.3 Hz), 3.08 (1H, dd, J=4.3, 17.3 Hz), 3.72 (3H, s), 4.59 (2H, d, J=5.5 Hz), 4.66 (1H, m), 5.23 (1H, dd, J=1.2, 10.4 Hz), 5.27-5.36 (1H, m), 5.82-5.97 (2H, m), 7.56 (1H, broad s).

Step 2

(S)N-Allyloxyearbonylaspartic Acid α-t-Butyl Ester, β-Methyl Ester

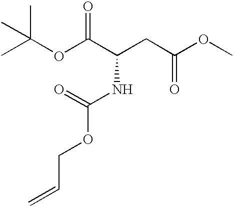

A mixture of (S)-N-allyloxycarbonylaspartic acid β-methyl ester (35.1 g), t-butyl acetate (150 ml) and 70% perchloric acid (11.0 ml) was stirred overnight at room temperature. The mixture was added dropwise to saturated NaHCO₃ solution and the product was extracted into ethyl acetate. The extracts were washed with brine, dried (MgSO₄) and concentrated to give the product as an oil (27.16 g, 62%).

MS (APCI+ve) M+H=288

¹H NMR (CDCl₃) 1.46 (9H, s), 2.81 (1H, dd, J=4.8, 16.8 Hz), 2.99 (1H, dd, J=4.4, 16.8 Hz), 3.69 (3H, s), 4.51 (1H, m), 4.58 (2H, d, J=5.3 Hz), 5.22 (1H, d, J=10.4 Hz), 5.31 (1H, dd, J=1.0, 17.0 Hz), 5.68 (1H, d, J=7.8 Hz), 5.92 (1H, m).

Step 3

(2R,3S) Methyl 3-allyloxycarbonylamino-4-t-butoxy-2-(2-naphthylmethyl)succinate

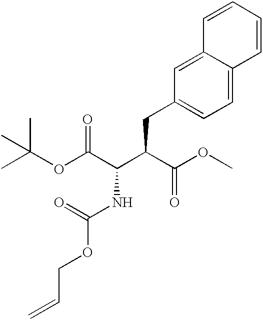

(S)-N-Allyloxycarbonylaspartic acid a-t-butyl ester, P-methyl ester (27.1 g, 94.3 mmol) in THF (200 ml) was added over 20 minutes to a stirred solution of LHMDS (207 ml of IM solution in THF, 0.207 mol) in THF (200 ml) at −70° C. under argon. The mixture was allowed to reach −40° C. over 1 hr and then re-cooled to −70° C. and 2-bromomethylnaphthalene (31.3 g, 0.142 mol) in THF (150 ml) was added. The mixture was stirred at −70° C. up to −40° C. over 6 hrs. Saturated ammonium chloride solution was added and the product was extracted into ethyl acetate. The extracts were washed with 10% aqueous citric acid, saturated NaHCO₃ solution and ten dried (MgSO₄) and concentrated. Flash chromatography on silica gel (ethyl acetate, dichloromethane, hexane) gave 18.53 g of pure product together with 11.36 g of slightly less pure material.

MS (ES+ve) M+Na=450

¹H NMR (CDCl₃) 1.43 (9H, s), 2.99 (1H, dd, J=7.5, 13.8 Hz), 3.21 (1H, dd, J=7.5, 13.8 Hz), 3.43 (1H, m), 3.63 (3H, s), 4.51 (1H, dd, J=3.5, 9.3 Hz), 4.62 (2H, d, J=5.5 Hz), 5.24 (1H, d, J=10.5 Hz), 5.35 (1H, d, J=17.2 Hz), 5.75 (1H, d, J=9.3 Hz), 5.94 (1H, m), 7.33 (1H, dd, J=1.5, 8.4 Hz), 7.40-7.49 (2H, m), 7.66 (1H, s), 7.66-7.83 (3H, m)

Step 4

(2R, 3S) 2-Allyloxycarbonylamino-4-t-butoxy-2-(2-naphthylmethyl)succinic acid

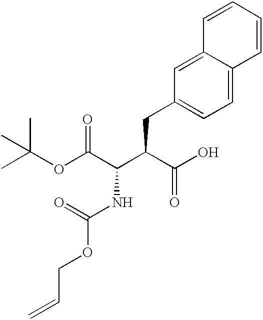

To a solution of (2R,3S) methyl 3-allyloxycarbonylamino-4-t-butoxy-2-(2-naphthylmethyl) succinate (16.37 g, 38.3 mmol) in 1,4-dioxan (100 ml) was added lithium hydroxide (2.41 g, 57.5 mmol) in water (120 ml). After stirring for 2 hrs at room temperature, the solvents were removed on the rotary evaporator and the residue was partitioned between ether and water. The aqueous layer was washed with ether and then acidified with 2M HCl and extracted with ethyl acetate. The organic extracts were washed with brine, dried (MgSO₄) and concentrated to afford the product as a gum (15.78 g, 100%).

MS (ES−ve) M−H=412

¹H NMR (CDCl₃) 1.40 (9H, s), 3.01 (1H, dd, J=8.2, 14.0 Hz), 3.28 (1H, dd, J=6.5, 14.0 Hz), 3.51 (1H, m), 4.51 (1H, dd, J=3.3, 9.5 Hz), 4.62 (2H, d, J=5.5 Hz), 5.24 (1H, d, J=10.3 Hz), 5.35 (1H, d, J=14.5 Hz), 5.83 (1H, d, J=9.8 Hz), 5.91 (1H, m), 7.36 (1H, dd, J=1.8, 8.5 Hz), 7.46 (2H, m), 7.69 (1H, s), 7.77-7.81 (3H, m), (some signals from a minor rotamer were also present).

Step 5

N′-[3S-Allyloxycarbonylamino-4-t-butoxy-2R-(2-naphthylmethyl)succinyl]-S-tert-leucine-N-methylamide

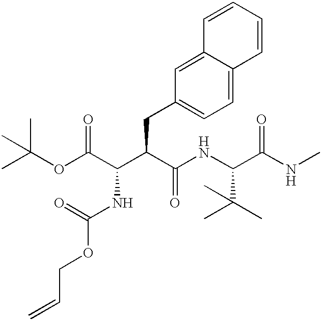

A mixture of (2R, 3S) 2-allyloxycarbonylamino-4-t-butoxy-2-(2-naphthylmethyl)succinic acid (10.60 g, 25.8 mmol), EDC (5.94 g, 31 mmol) and HOBT (4.74 g, 31.0 mmol) in DMF ((200 ml) was stirred at room temperature for 10 mins. (S)-t-Leucine methylamide (6.32 g, 35.0 mmol) and N-methyl morpholine (3.9 ml, 35.0 mmol) were added and the mixture was stirred at room temperature for 3 hrs. The DMF was evaporated and the mixture was partitioned between water and ethyl acetate. The product was extracted into ethyl acetate and the extracts were washed with 2M HCl, sodium bicarbonate solution and brine and then dried (MgSO₄) and concentrated. Chromatography on silica gel (40-50% ethyl acetate/hexane) gave the product as a white solid (11.16 g, 81%)

MS (ES+ve) M+H=540

¹H NMR (DMSO-d₆) 0.84 (9H, s), 1.40 (9H, s), 2.34 (3H, d, J=4.8 Hz), 2.84 (1H, dd, J=7.2, 13.6 Hz), 3.05 (1H, dd, J=8.4, 13.6 Hz), 3.44 (1H, m), 390 (1H, dd, J=5.6, 8.8 Hz), 4.10 (1H, d, J=9.6 Hz), 4.49 (2H, m), 5.21 (1H, dd, J=1.6, 10.4 Hz), 5.31 (1H, dd, J=1.6, 17.2 Hz), 5.92 (1H, m), 7.23 (1H, d, J=8.8 Hz), 7.36 (1H, dd, J=1.5, 8.5 Hz), 7.46 (2H, m), 7.61 (1H, s), 7.78 (3H, m), 7.85 (1H, m), 8.04 (1H, d, J=9.2 Hz), (some signals from a minor rotamer were also present).

Step 6

N′-[3S-Allyloxycarbonylamino-2R-(2-naphthylmethyl)succinyl]-S-tert-leucine-N-methylamide

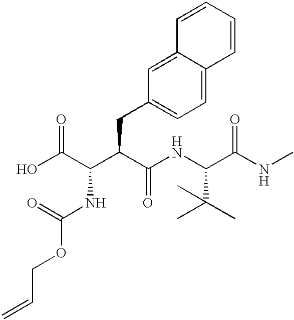

A solution of N′-[3S-allyloxycarbonylamino-4-t-butoxy-2R-(2-naphthylmethyl)succinyl]-S-tert-leucine-N-methylamide (13.06 g) in dichloromethane (100 ml) and trifluoroacetic acid (50 ml) was stirred at room temperature for 2.5 hrs. The solvents were removed and the residue was re-evaporated from chloroform (3×). The foamy solid was stirred in 1:1 ether-hexane for 2 hrs and then filtered and dried to give the product as a white powder (11.61 g, 99%).

MS (ES+ve) M+H=484, (ES−ve) M−H=482

¹H NMR (DMSO-d₆) 0.84 (9H, s), 2.36 (3H, d, J=4.4 Hz), 2.86 (1H, m), 3.06 (1H, m), 3.44 (1H, m), 3.93 (1H, dd, J=6.0, 8.8 Hz), 4.12 (1H, d, J=9.6 Hz), 4.50 (2H, m), 5.22 (1H, dd, J=1.5, 10.5 Hz), 5.32 (1H, dd, J=1.5, 17.5 Hz), 5.92 (1H, m), 7.07 (1H, d, J=9.2 Hz), 7.37 (1H, dd, J=1.2, 9.6 Hz), 7.47 (2H, m), 7.62 (1H, s), 7.72-7.81 (3H, m), 7.85 (1H, m), 7.96 (1H, d, J=9.0 Hz), 12.85 (1H, broad s), (some signals from a minor rotamer were also present).

Step 7

Attachment of N′-[3S-allyloxycarbonylamino-2R-(2-naphthylmethyl)succinyl]-S-tert-leucine-N-methylamide to Hydroxylamine Resin

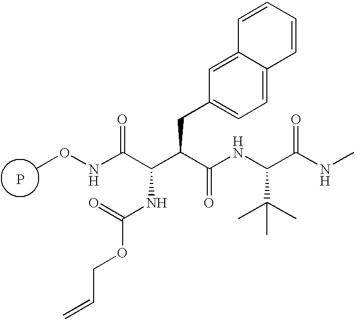

A mixture of SASRIN based hydroxylamine resin (7.54 g, 1 mmol/g, 7.54 mmol) and N′-[3 S-allyloxycarbonylamino-2R-(2-naphthyhnethyl)succinyl]-S-tert-leucine-N-methylamide (7.28 g, 15.1 mmol) in DMF (90 ml) was shaken at room temperature until the acid dissolved and then PyBop (7.86 g, 15.1 mmol), HOBT (2.31 g, 15.1 mmol) and N-methyl morpholine (1.66 ml, 15.1 mmol) were added and the mixture was shaken at room temperature overnight. The resin was collected by filtration and washed with DMF (3×), THF (2×), THF-water (1:1), THF, THF-water (1:1), THF (2×) and dichloromethane (4×). Drying under vacuum gave 10.13 g of resin.

Step 8

Removal of Alloc Group to Form Resin Bound N′-[3S-amino (N-hydroxyamino)-2R-(2-naphthylmethyl)succinyl]-S-tert-leucine-N-methylamide

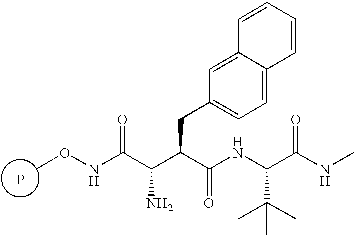

A mixture of the alloc protected resin (11.84 g, calculated loading 0.68 mmol/g, 8.05 mmol), acetic acid (2.42 ml, 42.3 mmol) and PdCl₂(PPh₃)₂ (565 mg, 0.805 mmol) in dichloromethane (120 ml) was stirred gently at room temperature and tri-n-butyltin hydride was added in 2 batches (7.0 ml and 2.75 ml, 5 minutes apart). After 10 minutes reaction in total, the resin was collected by filtration and washed with dichloromethane (3×), DMF (2×), 0.5% diethyldithiocarbamic acid sodium salt in DMF (2×), dichloromethane (2×), 10% triethylamine in dichloromethane (2×), dichloromethane (2×), methanol (3×) and dichloromethane (4×). Drying under vacuum gave 11.217 g of resin.

General Method for the Preparation of α-Sulfonamido Hydroxamic Acids Using the Myriad Personal Synthesiser.

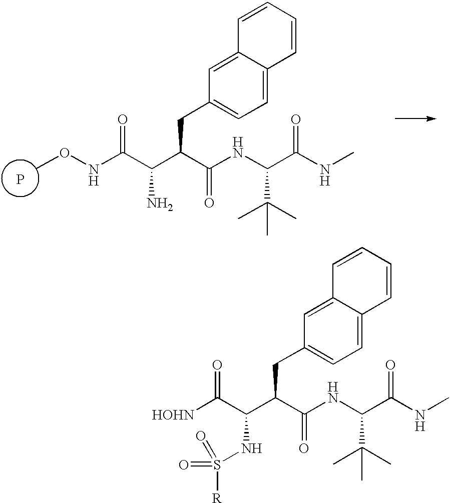

A Myriad solid phase reaction vessel was charged with the amino resin (200 mg, 0.142 mmol) and the resin was washed with dichloromethane-pyridine (2 ml, 1:0.6). The resin was then suspended in dichloromethane (1 ml) and pyridine (0.6 ml), stirred gently and a solution of sulfonyl chloride (1.1 ml of ˜0.71M solution in dichloromethane) was added. The mixture was stirred periodically during 4 hrs and the solution was then drained off. The resin was washed with dichloromethane (2×), DMF (2×), dichloromethane (2×), dichloromethane/methanol, methanol, dichloromethane/methanol, dichloromethane, dichloromethane/methanol and dichloromethane (3×).

The resin was suspended in 5% TFA in dichloromethane (2.8 ml) for 20 minutes and the mixture was then filtered and the resin washed with 5% TFA in dichloromethane (2 ml) and dichloromethane (2 ml). The combined filtrate and washings were concentrated on the rotary evaporator and then re-evaporated from chloroform (2×). Occasionally HPLC analysis indicated a small amount of unreacted amine remained and this was removed by stirring a THF solution of the product with an aldehyde resin followed by filtration and concentration. The products were then triturated with ether/hexane (2:1) and dried under vacuum.

### EXAMPLE 1

N -[3S-(Styrylsulfonamido)4-(N-hydroxyamino)-2R-(2-naphthylmethyl)succinyl]-S-tert-leucine-N-methylamide

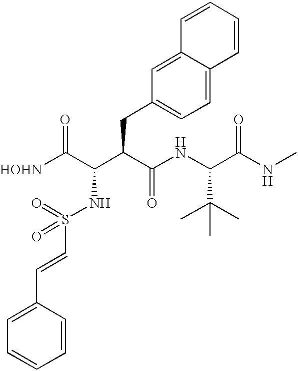

MS (ES+ve) M+H=581, (ES−ve) M−H=579

¹H NMR (DMSO-d₆) 0.82 (9H, s), 2.10 (3H, d, J=4.5 Hz), 2.66 (1H, m), 2.93 (1H, m), 3.07 (1H, m), 3.81 (1H, t, J=9.6 Hz), 4.04 (1H, d, J=9.6 Hz), 6.88 (1H, d, J=15.5 Hz), 7.19 (2H, m), 7.34 (1H, d, J=15.5 Hz), 7.40-7.53 (8H, m), 7.61 (2H, m), 7.72 (1H, d, J=8.5 Hz), 7.77 (1H, m), 7.82 (1H, m), 9.06 (1H, s), 10.96 (1H, s).

General Method for the Preparation of α-Amido Hydroxamic Acids Using the Myriad Personal synthesiser.

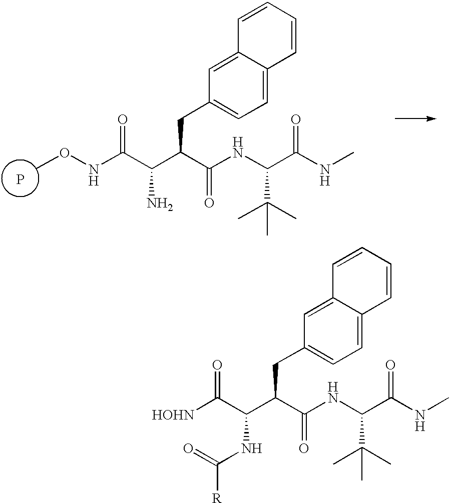

A Myriad solid phase reaction vessel was charged with the amino resin (200 mg, 0.142 mmol) and the resin was washed with DMF (2 ml). A solution of the carboxylic acid in DMF (1 mL of 0.71 M, 0.71 mmol, 5 eq.) was added, followed by a solution of diisopropyl carbodiimide in DMF (1.7 mL of 0.5 M, 0.852 mmol, 6 eq.) The mixture was stirred gently for 4 hours at room temperature, and then filtered and the resin was washed with DMF (2×2.8 mL), methanol (3×2.8 ml), dichloromethane (3×2.8 ml), methanol (2×2.8 mL) and dichloromethane (3×2.8 ml).

The progress of the each reaction was checked by the Kaiser test and by analytical HPLC after cleavage of the product from a few beads with TFA/dichloromethane (1:2). Any incomplete reactions were re-subjected to the reaction conditions.

The products were cleaved from the resin using a solution of 5% TFA in dichloromethane for 15 minutes at room temperature. The solution was then filtered and the resin washed with a few mL of 5% TFA in dichloromethane, then with dichloromethane. The filtrate and washings were combined, and concentrated under vacuum. Occasionally HPLC analysis indicated a small amount of unreacted amine remained and this was removed by stirring a THF solution of the product with an aldehyde resin followed by filtration and concentration. The products were then triturated with ether/hexane (2:1) and dried under vacuum.

### EXAMPLE 2

N′-[3S-(3,5-dichlorophenylcarboxamido)4(N-hydroxyamino)2R-(2-naphthylmethyl)succinyl]-S-tert-leucine-N-methylamide

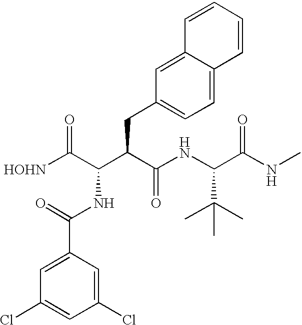

MS (ES+ve) M+H=587, (ES−ve) M−H 585,

¹H NMR (DMSO-d₆): 0.64 (9H, s), 2.16 (3H, d, J=4.4 Hz), 2.71 (1H, dd, J=3.2, 13.6 Hz), 3.04 (1H, dd, J=11.2; 13.6 Hz), 3.31 (1H, m), 4.02 (1H, d, J=10.0 Hz), 4.53 (1H, dd, J=8.4, 10.0 Hz), 7.20 (1H, d, J=10.0 Hz), 7.44 (3H, m), 7.51 (1H, s), 7.62. (1H, m), 7.76 (2H, m), 7.84 (2H, m), 7.92 (2H, s), 8.7(1H, d, J=8.4 Hz), 9.06 (1H, s), 11.13 (1H, s).

General Method for the Preparation of α-(Ar)alkylamino Hydroxamic Acids

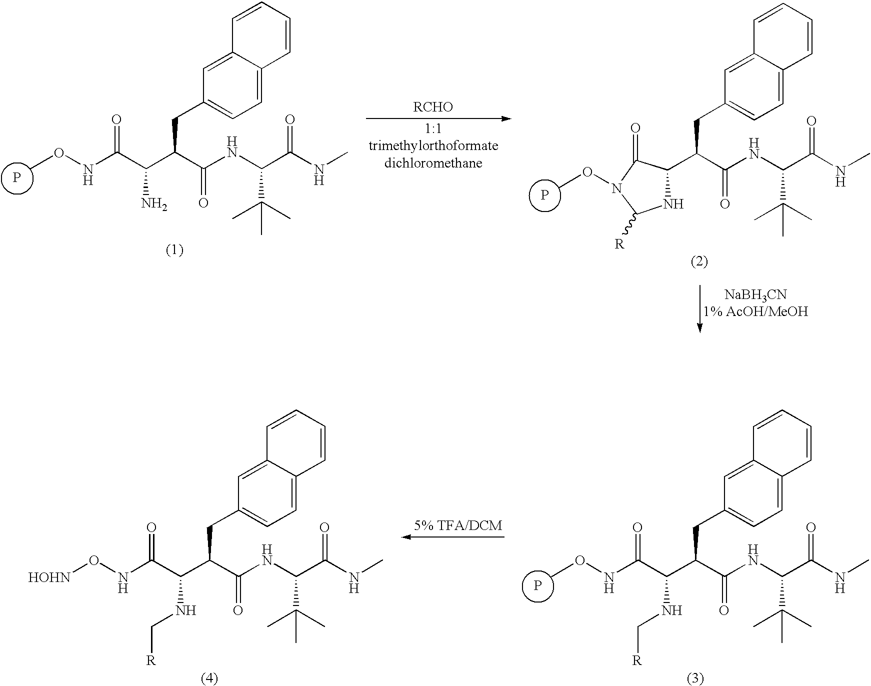

The α-amino resin (1) (200 mg, 0.142 mmol) in a 1:1 mixture of dichloromethane and trimethyl orthoformate (5 ml) was treated with the aldehyde (1.4 mmol, 10 equiv.) and the mixture was agitated gently for 1 h. The resin was then drained, washed with dichloromethane and then re-treated as described above but agitated for 2 h. The resin was drained and washed well with dichloromethane. (A small sample was cleaved with 5% TFA/dichloromethane and examined by HPLC and MS to confirm conversion to the imidazolidone). The resin (2) was then suspended in 1% acetic acid/methanol (5 ml) and treated with a solution of sodium cyanoborohydride (180 mg, 20 equiv.) in 1% acetic acid/methanol (5 ml). The mixture was gently stirred at room temperature for 3 days. The resin was then drained and washed sequentially with methanol (3×), 50% aqueous methanol (3×), methanol (3×), DMF (3×) and dichloromethane (4×). A small sample was cleaved with 5% TFA/dichloromethane and examined by HPLC and MS. If the starting imidazolidone was still present the reaction was repeated with several changes of reagent until complete reduction to the amine had occurred. In general this was from 7-14 days with up to 6 changes of reagent. After complete reaction was evident the resin was drained and washed as described above. The resulting resin bound (ar)alkyl amine derivative (3) was then cleaved by treatment with 5% TFA/dichloromethane (5 ml) at room temperature for 20 mins. The spent resin was filtered and the filtrate evaporated to dryness. The resulting residue was twice evaporated from toluene and then triturated with diethyl ether to afford the desired α-ar(alkyl)amino hydroxamic acid (4).

HPLC analysis was carried on a Vydac protein and peptide C18 column using a linear gradient of 10% to 90% of 70% acetonitrile/water in 0.1% TFA/water over 10 minutes with a flow rate of 1 ml/minute, unless otherwise stated. Detection was by UV at 220 nm.

### EXAMPLE 3

N′-[4(N-Hydroxyamino)-3S-[(furfur-2-yl)methylamino]-2R-(2-naphthylmethyl)succinyl]-S-tert-leucine-N-methylamide

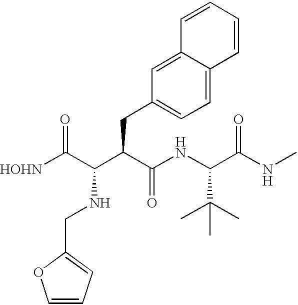

MS (ES+ve) M+H=495

HPLC retention time=7.93 mins.

¹H NMR (DMSO-d₆) 0.81 (9H, s), 2.13 (3H, d, J=4.5 Hz), 2.25 (1H, m), 2.64 (1H, dd, J=3, 14 Hz), 2.92 (1H, dd, J=13, 14 Hz), 3.05 (1H, m), 3.18 (1H, dd, J=10, 1 Hz), 3.43 (1H, dd, J=8, 14 Hz), 3.69 (1H, dd, J=4, 14 Hz), 4.07 (1H, d, J=9.5 Hz), 6.18 (1H, m), 6.34 (1H, m), 7.22 (1H, dd, J=1.6, 8.4 Hz), 7.29 (1H, q, J=4.5 Hz), 7.43 (2H, m), 7.48 (1H, s), 7.54 (1H, s), 7.57 (1H, d), 7.70-7.83 (3H, m), 9.02 (1H, s), 10.81 (1H, s).

General Method for the Preparation of α-(N-methyl-N-(ar)alkylamino) Hydroxamic Acids

The resin bound (ar)alkylamine (3) (0.114 mmol) was suspended in 2% acetic acid/dichloroethane (10 ml) and then treated with sodium triacetoxyborohydride (2.28 mmol, 20 equiv.). The mixture was stirred at room temperature for 5 minutes and then treated with a 37% aqueous solution of formaldehyde (2.28 mmol, 20 equiv.). Stirring was continued at room temperature for 4.25 h and then the resin was drained and washed sequentially with dichloroethane (3×), methanol (3×), 50% aqueous methanol (3×), DMF (3×), methanol (3×) and dichloromethane (4×). It was then cleaved with 5% TFA/dichloromethane (5 ml) at room temperature for 20 minutes. The spent resin was filtered and the filtrate was evaporated to dryness. The residue was twice evaporated from toluene and then triturated with diethyl ether to afford the N-methyl-N-(ar)alkylamino hydroxamic acid.

### EXAMPLE 4

N′-[4-(N-Hydroxyamino)3S-[N-propyl-N-methylamino]-2R-(2-naphthylmethyl)succinyl]-S-tert-leucine-N-methylamide

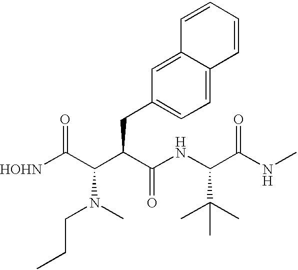

MS (ES+ve) M+H=485

HPLC retention time=8.73 mins.

### EXAMPLE 5

Preparation of N′-[4-(N-Hydroxyamino)-3S-(N,N-dimethylamino)-2R-(2-naphthylmethyl)succinyl]-S-tert-leucine-N-methylamide

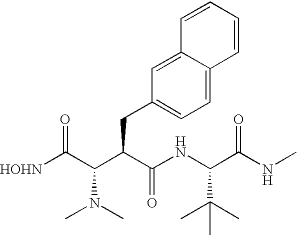

The resin bound amine (1) (200 mg, 0.142 mmol) was suspended in 2% acetic acid/dichloroethane (8 ml) and then treated with sodium triacetoxyborohydride (⁶00 mg). The mixture was stirred at room temperature for 5 minutes and then treated with a 37% aqueous solution of formaldehyde (0.23 ml, 20 equiv.). Stirring was continued at room temperature for 7 h and then the resin was drained and washed sequentially with dichloroethane (2×), methanol (3×), 50% aqueous methanol (3×), DMF (3×), methanol (3×) and dichloromethane. Cleavage of a small sample with 5% TFA/dichloromethane showed incomplete reaction so the resin was re-treated as above and stirred for 5.5 hrs. it was then filtered and washed as above to afford the resin bound dimethylamino derivative. Cleavage with 5% TFA/dichloromethane (4 ml) for 20 minutes at room temperature gave, after filtration, evaporation and trituration with diethyl ether, the title compound.

MS (ES+ve) M+H 443

HPLC retention time=5.30 mins. (Solvent system 30% to 70% of 70% acetonitrile/water in 0.1% TFA/water over 13 minutes with a flow rate of 1 ml/minute).

1. A compound of formula (I): 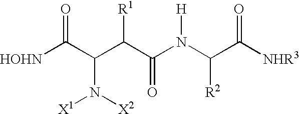
wherein:
X¹ is alkyl, sulphonyl or carboxy;
X² is hydrogen or alkyl;
R¹ is arylmethyl or heterocyclylmethyl;
R² is alkyl, alkenyl, aryl, cycloalkyl or cycloalkenyl; and
R³ is hydrogen, alkyl, alkenyl, alkynyl or aryl.

2. A compound according to claim 1, wherein X¹ is sulphonyl and X² is hydrogen, and/or R¹ is 1- or 2-naphthylmethyl; and/or R² is t-butyl; and/or R³ is hydrogen or methyl.

3. A compound according to claim 2, wherein each of X¹, X² and R¹ to R³ is selected from the group consisting of the values ascribed to it in the Examples hereinabove.

4. A compound according to claim 2, selected from the group consisting of the compounds described in the Examples hereinabove.

5. A compound according to any one of claims 1 to 4, which is a compound of formula (IA):

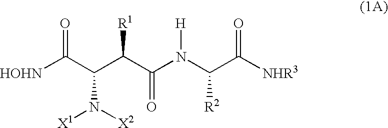

6. The use of a compound according to any one of the preceding claims for the production of a medicament for the treatment or prophylaxis of disorders such as allergy, inflammatory disorders and autoimmune disease in which the overproduction of s-CD23 is implicated.

7. A method for the treatment or prophylaxis of disorders such as allergy, inflammatory disorders and autoimmune disease in which the overproduction of s-CD23 is implicated, which method comprises the administration of a compound according to any one of claims 1 to 5 to a human or non-human mammal in need thereof.

8. A pharmaceutical composition for the treatment or prophylaxis of disorders such as allergy, inflammatory disorders and autoimmune disease in which the overproduction of s-CD23 is implicated which comprises a compound according to any one of claims 1 to 5 and optionally a pharmaceutically acceptable carrier therefor.

9. The use of a compound according to any one of claims 1 to 5 for the production of a medicament for the treatment or prophylaxis of conditions mediated by TNF, including, but not limited to, inflammation, fever, cardiovascular effects, haemorrhage, coagulation and acute phase response, cachexia and anorexia, acute infections, shock states, graft versus host reactions and autoimmune disease.

10. A method for the treatment or prophylaxis of conditions mediated by TNF, which method comprises the administration of a compound according to any one of claims 1 to 5 to a human or non-human mammal in need thereof.

11. A process for preparing a compound according to any one of claims 1 to 5, which process comprises
(a) deprotecting a compound of formula (II): 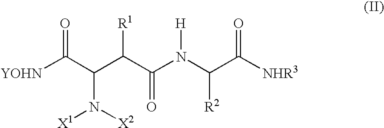
wherein X¹, X² and R¹ to R³ are as defined hereinabove, and Y is a protecting group such as benzyl or trimethylsilyl, or cleaving the compound of formula (II) from the Wang resin, where Y is the Wang resin residue, or
(b) reacting a compound of formula (M): 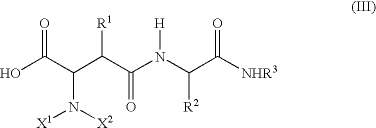
wherein X¹, X² and R¹ to R³ are as defined hereinabove, and any hydroxy group is optionally protected, with hydroxylamine or a salt thereof, or with Wang hydroxylamine resin followed by cleavage from the resin, or
(c) converting a compound of formula (I) to a different compound of formula (I) as defined hereinabove.

12. A compound of formula (II) as defined in claim 1 11.

13. A compound of formula (III) as defined in claim 11.
